(19) United States
(12) Patent Application Publication
(10) Pub. No.: US 2025/0241755 A1
(43) Pub. Date: Jul. 31, 2025

(54) SYSTEMS AND METHODS FOR CONTRACTING IMPLANTS

(71) Applicant: Edwards Lifesciences Innovation (Israel) Ltd., Caesarea (IL)

(72) Inventors: Kaamran Ahsan Syed, Irvine, CA (US); Brian Patrick Murphy, Irvine, CA (US); Meena Francis, Trabuco Canyon, CA (US)

(21) Appl. No.: 19/184,923

(22) Filed: Apr. 21, 2025

Related U.S. Application Data
(63) Continuation of application No. PCT/IB2023/060502, filed on Oct. 18, 2023.
(60) Provisional application No. 63/513,547, filed on Jul. 13, 2023.
(60) Provisional application No. 63/420,507, filed on Oct. 28, 2022.

(51) Int. Cl. A61F 2/24

(52) U.S. Cl. CPC A61F 2/2445; A61F 2/2466; A61F 2210/0014; A61F 2220/0016; A61F 2250/0001; A61F 2250/0058

(57) ABSTRACT

An implant comprises (i) first and second anchors, (ii) a tether extending between the first anchor and the second anchor, and (iii) a shape-memory band that extends alongside the tether, between the first anchor and the second anchor. A delivery assembly is adapted to (a) contract the implant at the heart, by applying energy to the shape-memory band, such that the shape-memory band draws the first and second anchors closer together, and (b) secure the implant in its contracted state by locking a stopper to the tether. Other implementations are also described.

### CROSS-REFERENCES TO RELATED APPLICATIONS

The present application is a Continuation of International Patent Application PCT/IB2023/060502 to Syed et al., filed Oct. 18, 2023, which published as WO 2024/089536, and which claims priority to:
Provisional U.S. Patent Application 63/420,507 to Syed et al., filed Oct. 28, 2022, and titled “Systems and methods for contracting implants;” and
Provisional U.S. Patent Application 63/513,547 to Syed et al., filed Jul. 13, 2023, and titled “Systems and methods for contracting implants.”

Each of the above applications is incorporated herein by reference in its entirety for all purposes.

### BACKGROUND

Some percutaneous techniques, including transluminal techniques such as transcatheter cardiac interventions, require components of an implant to be contracted once the implant is anchored to tissue of the heart. In some instances, contracting the components within the heart may exert significant forces on tissue anchors of the implant that are anchored to the tissue, e.g., undesirably pulling the anchors out of the tissue.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure can be included in the examples summarized here.

Methods, systems, devices, apparatuses, etc. for contracting an implant implanted at a real or simulated heart are disclosed. In some implementations, the implant is implanted at the heart, and a contracting force is then applied to the implant in order to contract tissue of the heart, e.g., to improve heart function.

In some implementations, it may be desirable to provide at least part of this contracting force from within the heart, rather than providing the entirety of the contracting force from outside the subject. For example, in some implementations in which the implant includes anchors which are anchored to the heart and connected via a tether extending between the anchors, when contracting the implant by drawing the anchors toward each other, rather than providing the entirety of the contraction force by pulling the tether from outside of the subject, it may be advantageous to provide at least part of the contracting force from within the heart, such as by causing another component of the implant to draw the anchors toward each other. For example, this may advantageously allow less force to be applied to the tether during contraction, and/or may allow for more controlled and/or reliable contraction of the implant.

In some implementations, the implant is an annuloplasty implant. In some implementations, the implant (e.g., annuloplasty implant) includes a plurality of anchors anchorable around an annulus of the heart. In some implementations, a tether (e.g., a wire, a contracting member, a string and/or a line) extends between at least a first anchor and a second anchor of the plurality of anchors.

In some implementations, a shape-memory band extends between the first anchor and the second anchor, e.g., alongside the tether.

In some implementations, the anchors are first anchored at the annulus, and electrical energy is then applied to the shape-memory band (e.g., via a delivery tool that is electrically connectable to the band) to temporarily contract the implant. Responsively to the electrical energy, the shape-memory band contracts, thereby reducing the distance between the first anchor and the second anchor, e.g., by pulling the anchors towards each other.

In some implementations, the band remains contracted while the electrical energy continues to be applied. In some implementations, while the electrical energy continues to be applied to the shape-memory band, a lock is locked to the tether, thus maintaining the implant in the contracted state by maintaining the reduced distance between the anchors. The lock may already be present before the electrical energy is applied, or can be advanced to the implant while the electrical energy continues to be applied.

As described herein, electrical augmentation of the contraction of the implant may reduce pulling force required to be applied to the tether from outside the heart (e.g., by a user manipulating a handle or controls thereof) in order to contract the implant.

In accordance with some implementations, a system (e.g., useable and/or for use at a real or simulated heart) includes an implant. In some implementations, the implant includes a first anchor and a second anchor, and a tether extending between the first anchor and the second anchor. In some implementations, a shape-memory band extends alongside the tether, between the first anchor and the second anchor.

In some implementations, the system further includes a delivery assembly, adapted to contract the implant at the heart. In some implementations, the delivery assembly is adapted to contract the implant at the heart by applying energy to the shape-memory band, such that the shape-memory band draws the first anchor and the second anchor closer together.

In some implementations, the delivery assembly is further adapted to secure tension in the implant by locking a stopper to the tether.

In some implementations, the implant is an annuloplasty implant.

In some implementations, each of the first anchor and the second anchor includes a helical tissue-engaging element.

In some implementations, the delivery assembly includes an anchor driver, adapted to drive the first anchor into tissue of the heart.

In some implementations, the shape-memory band is a nitinol band.

In some implementations, the shape-memory band is fixed to both the first anchor and the second anchor.

In some implementations, the first anchor is drivable into tissue of the heart while the second anchor is disposed within the delivery assembly, at the heart.

In some implementations, the implant is a first implant, the system further includes a second implant, and the delivery tool is configured to arrange the first implant and the second implant around an annulus of the heart.

In some implementations, the first anchor and the second anchor are anchors of a plurality of anchors of the implant. In some implementations, both the tether and the shape-memory band extend between the plurality of anchors.

In some implementations, the stopper is a bead, slidable over and along the tether.

In some implementations, the delivery assembly includes a locking tool configured to advance the stopper over and along the tether.

In some implementations, the shape-memory band is heat-set to assume a contracted shape upon application of the energy.

In some implementations, the contracted shape is a zigzag shape.

In some implementations, the contracted shape is a serpentine shape.

In some implementations, the contracted shape is a helical shape.

In some implementations, the shape-memory band is adapted to return toward a relaxed state responsively to cessation of application of the energy to the shape-memory band.

In some implementations, the first anchor and the second anchor are a first pair of anchors, the implant including multiple pairs of anchors. In some implementations, the shape-memory band is a first shape-memory band, the implant including multiple shape-memory bands, each of the shape-memory bands connecting the anchors of a respective pair. In some implementations, the tether extends between all of the anchors of the multiple pairs. In some implementations, the tether extends between only some (not all) of the anchors of the multiple pairs.

In some implementations, the implant defines a band-free gap in which no shape-memory band connects the first pair of anchors with a second pair of anchors, the second pair being adjacent to the first pair within the implant.

In some implementations, the stopper is a first stopper of a plurality of stoppers. In some implementations, the delivery assembly is adapted to iteratively secure tension in the implant by, for each pair of anchors: (i) applying the energy to the shape-memory band that connects the anchors of the pair, and (ii) securing tension in a segment of the tether between the anchors of the pair by locking a respective stopper of the plurality of stoppers to the tether.

In some implementations, the delivery assembly is configured to apply the energy to each of the shape-memory bands wirelessly.

In some implementations, the shape-memory band is heat-set to transition toward a contracted shape at a temperature greater than 37.5 degrees Celsius.

In some implementations, the shape-memory band is heat-set to transition toward the contracted shape at a temperature greater than 45 degrees Celsius.

In some implementations, the shape-memory band is heat-set to transition toward the contracted shape at a temperature greater than 50 degrees Celsius.

In some implementations, the shape-memory band is heat-set to transition toward the contracted shape at a temperature less than 60 degrees Celsius.

In some implementations, for each of the first anchor and the second anchor, the anchor includes an anchor head, and a collar that circumscribes and is rotatable about the anchor head. In some implementations, the shape-memory band is attached to the collar of the first anchor and the collar of the second anchor.

In some implementations, each collar defines an eyelet, and the tether extends through the eyelet of the first anchor and the eyelet of the second anchor.

In some implementations, the tether is slidable through the eyelet of the second anchor.

In some implementations, the first anchor includes a terminal on the collar, electrically connectable to the delivery assembly, and configured to conduct the energy from the delivery assembly to the shape-memory band.

In some implementations, the delivery assembly includes a catheter that is configured to transluminally deliver the implant to the heart.

In some implementations, the catheter is configured to transfemorally deliver the implant to the heart.

In some implementations, the first anchor includes a terminal, electrically connected to the shape-memory band, and via which the delivery system is configured to apply the energy to the shape-memory band.

In some implementations, the first anchor includes a tissue-engaging element, configured to anchor the first anchor to the tissue by being driven into the tissue, and the terminal is electrically isolated from the tissue-engaging element.

In some implementations, the system further includes an extracorporeal power generator, and a conductor adapted to electrically connect the terminal to the power generator.

In some implementations, the power generator is adapted to apply the energy to the shape-memory band via electrical connection between the conductor and the terminal.

In some implementations, the conductor is adapted to extend, from the terminal, through the delivery assembly, and out of the subject, where the conductor is connected to the power generator.

In some implementations, the delivery assembly further includes a wireless transmitter, adapted to transmit the energy wirelessly. In some implementations, the implant includes a receiver, adapted to receive the wireless energy and to transfer at least part of the energy to the shape-memory band.

In some implementations, the wireless transmitter is adapted to transmit the energy electromagnetically.

In some implementations, the first anchor and the second anchor are a first pair of anchors, and the implant includes multiple pairs of anchors. In some implementations, the shape-memory band can be a first shape-memory band, and the implant can include multiple shape-memory bands, each of the shape-memory bands connecting the anchors of a respective pair.

In some implementations, the implant further includes a hub with which the delivery assembly is engageable.

In some implementations, the hub can include a switch that is operable by the delivery assembly to select a subset of the shape-memory bands to which to apply the energy.

In some implementations, the subset of shape-memory bands includes only a single shape-memory band.

In some implementations, the subset of shape-memory bands includes multiple shape-memory bands.

In some implementations, the hub includes a hub terminal, and the delivery assembly is adapted to engage the hub terminal in a manner that places the delivery assembly in electrical connection with the hub.

In some implementations, the delivery assembly is adapted to operate the switch by rotating the switch.

In some implementations, the switch is a rotary switch.

In some implementations, the rotary switch has at least four switching positions.

In some implementations, for each subset of shape-memory bands, the implant includes a respective terminal, electrically connected to the respective subset. In some implementations each terminal is electrically connected to the switch via a corresponding wire. In some implementations, operation of the switch to select the subset electrically connects the delivery assembly to the respective terminal of the subset.

In some implementations, the system further includes an extracorporeal power generator, adapted to apply the energy to the subset of shape-memory bands via electrical connection between the power generator and the respective terminal of the subset.

In some implementations, each wire is electrically isolated from the other wires.

In some implementations, the delivery assembly is adapted to iteratively contract each subset by sequentially delivering a burst of energy to each subset.

In accordance with some implementations, a method (e.g., useable and/or for use at a real or simulated heart) includes anchoring an implant to tissue of the heart by anchoring a first anchor of the implant to the tissue, and a second anchor of the implant to the tissue, a tether of the implant extending between the first anchor and the second anchor.

In some implementations, the method includes, subsequently, reducing a distance between the first anchor and the second anchor by applying energy to a shape-memory band that extends, alongside the tether, between the first anchor and the second anchor.

In some implementations, the method includes subsequently fixing the reduced distance by locking a stopper to the tether.

In some implementations, the method further includes ceasing to apply the energy subsequently to fixing the reduced distance.

In some implementations, reducing the distance includes reducing the distance without applying tension to the tether.

In some implementations, the implant is an annuloplasty implant, and anchoring the first anchor and the second anchor to tissue of the heart includes anchoring the first anchor and the second anchor to tissue of the annulus.

In some implementations, applying the energy to the shape-memory band includes transmitting the energy to the implant wirelessly.

In some implementations, applying the energy to the shape-memory band includes applying the energy to the shape-memory band such that the shape-memory band becomes heated to a temperature greater than 37.5 degrees C.

In some implementations, anchoring the first anchor and the second anchor to tissue of the heart includes driving a tissue-engaging element of the first anchor and a tissue-engaging element of the second anchor into the tissue.

In some implementations, the method further includes sliding the stopper over and along the tether such that the stopper abuts the second anchor.

In some implementations, anchoring the first anchor to the tissue includes anchoring the first anchor to the tissue whilst the second anchor is disposed within a distal part of a delivery assembly that is disposed within the heart.

In some implementations, the method further includes, subsequently to locking the stopper to the tether, trimming an excess of the tether.

In some implementations, the implant is a first implant, and the method includes implanting multiple implants in an arrangement along the tissue.

In some implementations, the first anchor and the second anchor are anchors of a plurality of anchors of the implant. In some implementations, anchoring the implant to the tissue includes anchoring the implant along the tissue while both the tether and the shape-memory band extend between the plurality of anchors.

In some implementations, the method further includes applying tension to the tether prior to locking the stopper to the tether.

In some implementations, applying the tension to the tether includes applying the tension to the tether subsequently to commencing applying the energy to the shape-memory band.

In some implementations, the first anchor and the second anchor are a first pair of anchors, the implant including multiple pairs of anchors. In some implementations, the shape-memory band is a first shape-memory band, the implant including multiple shape-memory bands, each of the shape-memory bands connecting the anchors of a respective pair.

In some implementations, the method includes implanting the implant along the tissue while the tether extends between all of the anchors of the multiple pairs.

In some implementations, anchoring the implant to the tissue includes anchoring the implant to the tissue in a manner that defines a band-free gap in which no shape-memory band connects the first pair of anchors with a second pair of anchors of the implant, the second pair being adjacent to the first pair within the implant.

In some implementations, the stopper is a first stopper of a plurality of stoppers. In some implementations, the method includes iteratively securing tension in the implant by, for each pair of anchors: (i) applying the energy to the shape-memory band that connects the anchors of the pair, and (ii) securing tension in a segment of the tether between the anchors of the pair by locking a respective stopper of the plurality of stoppers to the tether.

In some implementations, the method includes transluminally delivering the implant to the heart.

In some implementations, transluminally delivering the implant to the heart includes transfemorally delivering the implant to the heart.

In some implementations, applying the energy to the shape-memory band includes applying the energy to the shape-memory band such that shape-memory band transitions toward a contracted shape.

In some implementations, the method further includes, subsequently to locking the stopper to the tether, ceasing to apply the energy to the shape-memory band, such that the shape-memory band returns toward a relaxed state.

In some implementations, the implant includes multiple pairs of anchors, the first anchor and the second anchor being one of the pairs of anchors. In some implementations, the shape-memory band can be a first shape-memory band, the implant comprising multiple shape-memory bands, each of the shape-memory bands connecting the anchors of a respective pair.

In some implementations, the implant can further include a hub. In some implementations, the hub can comprise a switch.

In some implementations, the method can further comprise, prior to applying the energy to the first shape-memory band, selecting a subset of the multiple shape-memory bands by operating the switch, the subset including the first shape-memory band.

In some implementations, reducing the distance between the first anchor and the second anchor by applying energy to the first shape-memory band comprises reducing the distance between the first anchor and the second anchor by applying the energy to the selected subset of shape-memory bands.

In some implementations, the switch is a rotary switch, and operating the switch comprises rotating the rotary switch.

The above method(s) can be performed on a living animal or on a non-living simulation, such as on a cadaver, cadaver heart, simulator (e.g., with the body parts, tissue, etc. being simulated), etc.

In accordance with some implementations, a method (e.g., useable and/or for use at a real or simulated heart) includes implanting an implant at the heart by securing a plurality of anchors of the implant to tissue of the heart, the anchors connected together via (i) a shape-memory band, and/or (ii) a tether.

In some implementations, the method includes transitioning the implant towards a contracted state by applying electrical energy to the shape-memory band, such that the anchors move closer together.

In some implementations, the method includes locking the implant in the contracted state by applying a stopper to the tether.

The above method(s) can be performed on a living animal or on a non-living simulation, such as on a cadaver, cadaver heart, simulator (e.g., with the body parts, tissue, etc. being simulated), etc.

In accordance with some implementations, a method (e.g., useable and/or for use at a real or simulated heart) includes anchoring, to tissue of the heart, a first anchor and a second anchor, a portion of a tether extending between the first anchor and the second anchor, the portion having a length. In some implementations, the method includes, subsequently, reducing the length of the portion of the tether, facilitated by applying electrical energy to a shape-memory band that extends, alongside the tether, between the first anchor and the second anchor.

In some implementations, the method includes subsequently fixing the reduced length by locking a stopper to the tether.

In accordance with some implementations, a system and/or an apparatus (e.g., useable and/or for use at a real or simulated heart) including an implant includes multiple shape-memory bands, configured such that application of energy to a shape-memory band contracts a respective portion of the implant.

In some implementations, the hub can include a hub terminal, via which the energy is applied, and/or a switch that is operable to select a shape-memory band to which to direct the energy.

In some implementations, the system/apparatus further includes a delivery assembly configured to deliver the implant into a subject, the delivery assembly comprising a shaft that is adapted to engage the hub terminal in a manner that places the delivery assembly in electrical connection with the hub. In some implementations, the hub can be adapted to operate the switch to select the shape-memory band.

In some implementations, the shaft is flexible, and is adapted to be transluminally advanced to a tissue of a subject.

In some implementations, the shaft is adapted to operate the switch by rotating the switch.

In some implementations, the multiple shape-memory bands are grouped into multiple subsets of shape-memory bands. In some implementations, for each subset of shape-memory bands, the implant can include a respective terminal, electrically connected to the respective subset.

In some implementations, each terminal can be electrically connected to the switch via a corresponding wire. In some implementations, operation of the switch to select the subset can electrically connect the shaft to the respective terminal of the subset.

In some implementations, the system/apparatus further includes an extracorporeal power generator, electrically connected to the shaft, and adapted to apply the energy to the subset of shape-memory bands via the electrical connection between the shaft and the respective terminal of the subset.

In some implementations, each wire is electrically isolated from the other wires.

In some implementations, the switch is a rotary switch.

In some implementations, the rotary switch has at least four switching positions.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, devices, apparatuses, components, etc. can be sterilized (e.g., with heat, radiation, ethylene oxide, hydrogen peroxide, etc.) to ensure they are safe for use with patients, and the above methods can comprise (or additional methods consist of) sterilization of one or more systems, devices, apparatuses, components, etc. herein (e.g., with heat, radiation, ethylene oxide, hydrogen peroxide, etc.).

The concepts herein will be more fully understood from the following detailed description of example implementations thereof, taken together with the drawings.

### BRIEF DESCRIPTION OF THE DRAWINGS

FIGS. 1A-E are schematic illustrations of a system comprising an implant, and techniques for contracting the implant, in accordance with some implementations;

FIGS. 2, 3, and 4 are schematic illustrations of different uses and/or variants of the implant implanted around an annulus of a real or simulated heart, in accordance with some implementations; and

FIGS. 5 and 6A-C are schematic illustrations of a hub on an implant that is operable to select a subset of the implant to which to apply energy.

### DETAILED DESCRIPTION

The present application relates to methods and systems for contracting an implant implanted at a heart. In some implementations, the implant is implanted at a heart (e.g., a heart of a living subject and/or a heart of a simulation), and a contracting force is then applied to the implant in order to contract tissue of the heart, e.g., to improve heart function. In some implementations, it may be desirable to provide at least part of this contracting force from within the heart of the subject, rather than providing the entirety of the contracting force from outside the subject.

For example, in some implementations in which the implant includes anchors which are anchored to the heart and connected via a tether extending between the anchors, when contracting the implant by drawing the anchors toward each other, rather than providing the entirety of the contraction force by pulling the tether from outside of the subject, it may be advantageous to provide at least part of the contracting force from within the heart, such as by causing another component of the implant to draw the anchors toward each other. For example, this may advantageously allow less force to be applied to the tether during contraction, and/or may allow for more controlled and/or reliable contraction of the implant.

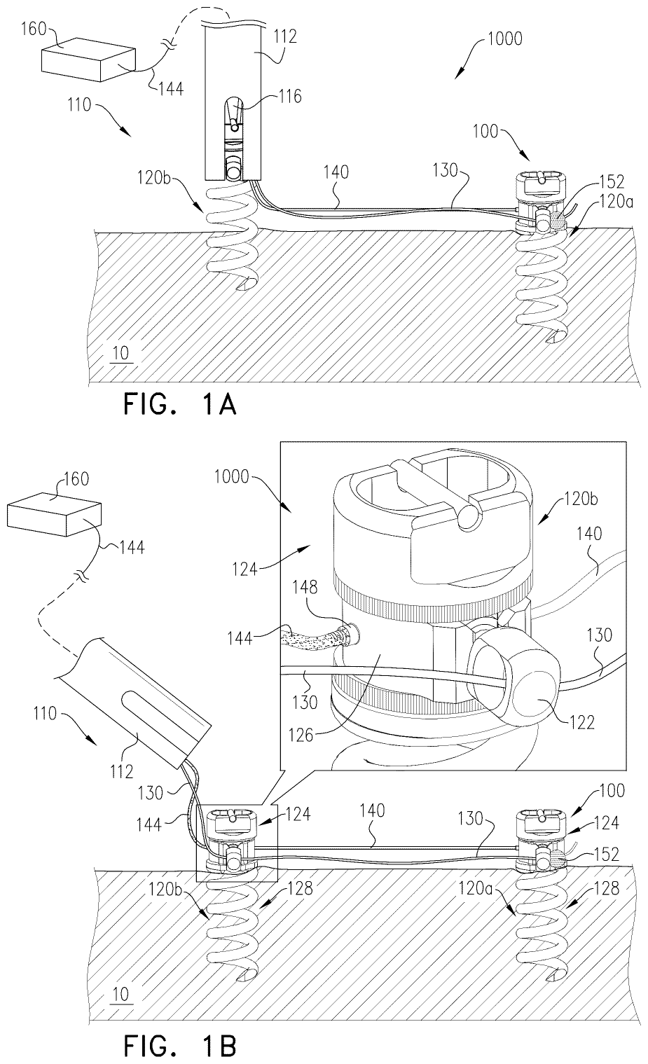

Reference is now made to FIGS. 1A-E, which are schematic illustrations of a system 1000, in accordance with some implementations. System 1000 comprises an implant 100, and can further comprise a delivery assembly 110, adapted to transluminally (e.g., transfemorally) deliver and implant implant 100 at a heart. Delivery assembly 110 typically comprises a catheter 112, an anchor driver 116, and a locking tool 114.

Implant 100 comprises multiple anchors 120, e.g., a first anchor 120a and a second anchor 120b. Each anchor 120 has a head 124 and a tissue-engaging element 128 extending distally from the head. Tissue-engaging elements 128 are shown as helical and/or screw-in tissue-engaging elements, however any other type of tissue-engaging element may be used, e.g., darts, staples, or clips. Implant 100 further comprises a tether 130 that extends between anchors 120a and 120b. Tether 130 may be a string, a rope, a line, a suture, a cable, and/or a wire.

In some implementations, implant 100 is an annuloplasty implant, adapted to be implanted around an annulus 10 of the heart. In some implementations, annulus 10 is a mitral annulus. In some implementations, annulus 10 is a tricuspid annulus.

System 1000 further comprises a shape-memory band 140, that extends between anchors 120a and 120b, e.g., alongside tether 130. Shape-memory band 140 may be made of nitinol or any other shape-memory alloy. Shape-memory band 140 may comprise a piezoelectric material. Shape-memory band 140 is adapted (e.g., heat set) to assume a contracted state upon heating of the band, e.g., via application of electrical energy (e.g., a radio-frequency (RF) current). In some implementations, shape-memory band 140 is adapted to return toward a relaxed state upon cooling, e.g., upon cessation of the application of electrical energy to the band. Shape-memory band 140 may be configured (e.g., heat set) such that its transition temperature (i.e., the temperature at which it transitions toward its contracted state) is greater than 37.5 degrees C. (e.g., greater than 40 degrees C., such as greater than 42 degrees C.) and/or less than 60 degrees C. (e.g., less than 50 degrees C.).

In some implementations, each anchor head 124 comprises a collar 126 (e.g., a ring) that circumscribes and is rotatable about the anchor head, e.g., by being rotatably coupled to the anchor, such as by being rotatably coupled to another component of head 124 that is fixedly coupled to tissue-engaging element 128 of the anchor. In some implementations, anchors 120, and/or anchor head 124, and/or collar 126 thereof is similar to as described in WO 2021/084407 to Kasher, mutatis mutandis, which is incorporated herein by reference in its entirety. In some implementations, an eyelet 122 is mounted on each collar 126 of each anchor head, and tether 130 extends through these eyelets. In some implementations, tether 130 is slidable through the eyelets—or at least through the eyelet of anchor 120b.

In some implementations, shape-memory band 140 is fixed to the head of each of anchors 120a and 120b (e.g., to collar 126 thereof), such that transitioning the band toward its contracted state pulls the anchors toward each other.

In some implementations, connecting eyelets 122 and band 140 to rotatable collar 126 advantageously allows tissue-engaging element 128 to be screwed into the tissue without undesirably winding or tangling tether 130 or band 140 around anchor 120 (e.g., around head 124). For example, collar 126 may remain stationary with respect to the tissue while tissue-engaging element 128 is rotated with respect to the tissue.

In some implementations, a terminal 148 is mounted on at least one of anchors 120a and 120b, via which electrical energy is transferred to shape-memory band 140. For example, terminal 148 may be electrically and/or mechanically connected to shape-memory band 140, e.g., via a conductive element of anchor head 124, for example, by both the terminal and the band being connected to collar 126. In some implementations, terminal 148 is electrically isolated from tissue-engaging element(s) 128, e.g., in order to prevent the electrical energy from being transmitted to tissue of the heart.

FIG. 1A shows anchor driver 116 driving second anchor 120b into tissue of annulus 10, with first anchor 120a already having been anchored to the tissue. First anchor 120a may be delivered to the heart with a first stopper 152 positioned on tether 130 distally to the anchor. This may prevent anchor 120a from sliding off tether 130. During driving of the anchors into the tissue, anchor driver 116 may be housed in catheter 112, e.g., as shown in FIG. 1A. In some implementations, the driving of anchors 120 is as described, mutatis mutandis, in WO 2021/084407 to Kasher. In some implementations, anchor driver 116 is as described in US Patent Application Publication US 2014/0309661 to Sheps et al., which is incorporated herein by reference in its entirety.

In some implementations, anchors 120a and 120b are driven into the tissue of the heart while tether 130 and/or shape-memory band 140 already extends therebetween. In some implementations, during implantation of implant 100, tether 130 extends, from the anchors, through vasculature of the subject (e.g., via catheter 112), e.g., to outside of the subject. Once both anchors 120a and 120b have been driven into the tissue, anchor driver 116 can be withdrawn from the heart (e.g., via catheter 112) and out of the subject (FIG. 1B). FIG. 1B shows band 140 in its relaxed state.

In some implementations, a conductor 144 (e.g., a conductive wire) extends, from an extracorporeal power generator 160 to terminal 148, e.g., via catheter 112. Power generator 160 is adapted to apply, via conductor 144 and terminal 148, electrical energy to shape-memory band 140 in order to heat to the band such that the band transitions toward its contracted state. FIG. 1C shows electrical energy being applied to band 140, and the band having responsively transitioned towards its contracted state. As shown, this draws anchors 120a and 120b towards each other (illustrated with arrows), thus contracting the implant. In some implementations, during application of the electrical energy to shape-memory band 140, a closed electrical circuit (e.g., a closed loop) exists between power generator 160 and terminal 148, e.g., by conductor 144 extending, from the power generator to the terminal (e.g., via catheter 112), and from the terminal, back through the catheter to the power generator.

In the example shown, in its contracted state, band 140 has a zigzag shape. However, it is to be understood that the scope of the disclosure includes band 140 being configured to have other contracted shapes that draw anchors 120 toward each other. Nonlimiting examples of such other contracted shapes include a serpentine shape and a coil (e.g., helical) shape.

While shape-memory band 140 remains in its contracted state (e.g., while electrical energy continues to be applied to the shape-memory band), slack that may exist in tether 130 can be taken up (e.g., by pulling the tether proximally), and the tether can then be locked with respect to anchor 120b, e.g., using a second stopper 154 (FIG. 1D). Stopper 154 may be positioned such that it abuts anchor 120b, e.g., such that it abuts eyelet 122 of anchor 120b. In some implementations, although the slack is taken up, no substantial additional tension is applied to tether 130 prior to locking.

In some implementations, not only is slack taken up, but additional tension is applied to tether 130 prior to locking. Furthermore, in some implementations the electrical energy is applied substantially concurrently with the application of tension to tether 130, e.g., the contraction of band 140 simply augments the contraction provided by tensioning the tether.

As described hereinabove, the contraction of band 140 may advantageously allow for less (or even substantially no) pulling force to be applied to tether 130, compared to a similar system in which such electrical augmentation of contraction is not provided.

Additionally or alternatively, such electrical augmentation may enhance control and/or reliability of contraction of implant 100. In some implementations, during the take-up of slack in tether 130 (and/or the tensioning of the tether), locking tool 114 is adapted to apply a reference force to anchor 120b, e.g., by pushing against the anchor (e.g., via stopper 154) while the tether is pulled proximally.

FIG. 1C illustrates that the electrical energy may be applied prior to advancement of tool 114 and/or stopper 154, but is primarily intended to illustrate that the application of the electrical energy may be independent of the tool and/or of any tensioning of tether 130. However, it is to be noted that, in some implementations, the state shown in FIG. 1C may not exist during the procedure, e.g., tool 114 and/or stopper 154 may be present at anchor 120b prior to the application of the electrical energy.

In some implementations, the stopper is advanced using locking tool 114. In some implementations, stopper 152 and/or stopper 154 are variants of, or substantially identical to, one or more of the stoppers described in WO 2021/084407 to Kasher et al.

In some implementations, tether 130 is then cut (e.g., trimmed), e.g., proximally to stopper 154, and delivery assembly 110 is withdrawn, leaving implant 100 implanted within the heart (FIG. 1E). Application of the electrical energy to band 140 may be stopped prior to or subsequently to the cutting of tether 130. FIG. 1E shows band 140 having responsively returned toward its relaxed state—although it has more slack than in FIG. 1B because anchors 120 have become closer to each other.

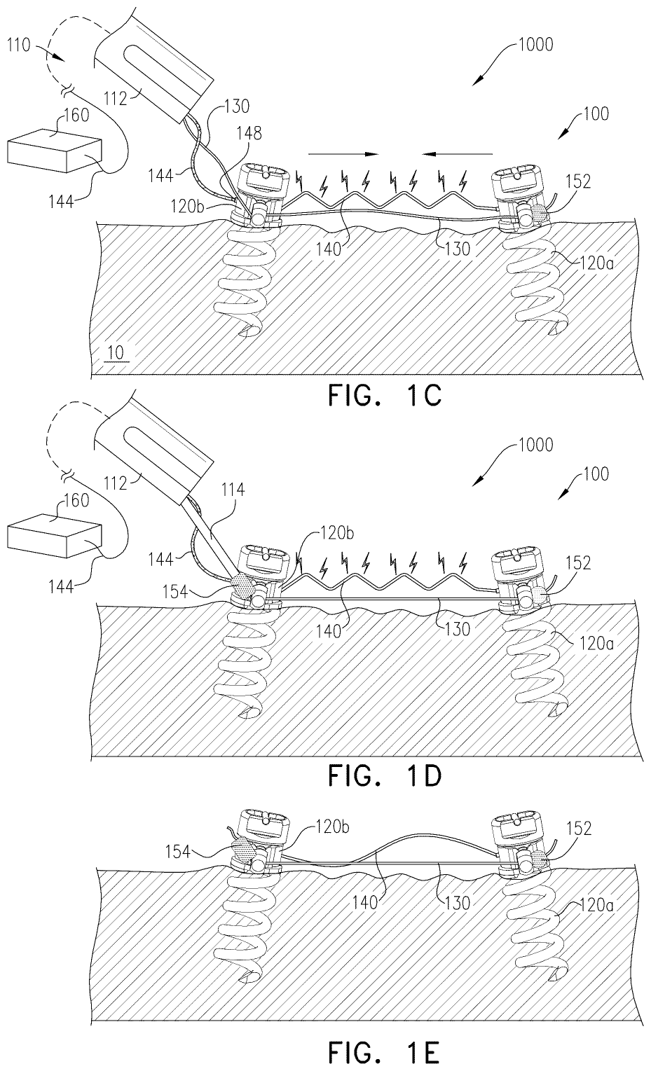

Reference is now made to FIGS. 2, 3, and 4, which show different uses and/or variants of implant 100 implanted around an annulus 10 of a heart, in accordance with some implementations. Each of the implants shown in FIGS. 2-4 may be adapted for use with system 1000 (or a variant thereof), e.g., may be deliverable via delivery assembly 110 (or a variant thereof). In some implementations, the implants shown in FIGS. 2-4 are contracted using the same general technique as described with reference to FIGS. 1A-E, e.g., electrical energy is applied to shape-memory band(s) of the implant in order to contract, or facilitate contraction of, the implant, and the implant is then locked in a contracted state by locking tension within tether(s) of the implant.

FIG. 2 shows an example implant arrangement 200 comprising multiple implants 100. As described hereinabove, in some implementations, each implant 100 comprises a pair of anchors 120, and a tether 130 extending between the pair. Additionally, in some implementations, each implant 100 comprises a shape-memory band 140, extending between the anchors, alongside tether 130.

In some implementations, each implant 100 of implant arrangement 200 can be anchored and/or contracted as described hereinabove with reference to FIGS. 1A-E, mutatis mutandis.

In some implementations, each implant 100 of implant arrangement 200 may be anchored independently of the others, e.g., the implants of the implant arrangement may be anchored sequentially.

In some implementations, each implant 100 of implant arrangement 200 may be contracted independently of the others, e.g., the implants of the implant arrangement may be contracted sequentially.

In some implementations, each implant 100 is contracted immediately following its implantation and prior to the implantation of the next implant of the implant arrangement. In some implementations, multiple (e.g., all) of the implants of the implant arrangement are anchored, and then subsequently contracted in concert, e.g., such that the contraction of each of the implants may be adjusted in coordination with the contraction of other implants of the implant arrangement.

In some implementations, each implant 100 of implant arrangement 200 has a pair of stoppers 152 and 154 positioned on opposite ends of its respective tether 130, in order to maintain the tension in each tether. In some implementations, the pair of anchors 120 of each implant 100 are delivered together, e.g., the first anchor of the pair is delivered while the second anchor is positioned within delivery assembly 110, at the heart.

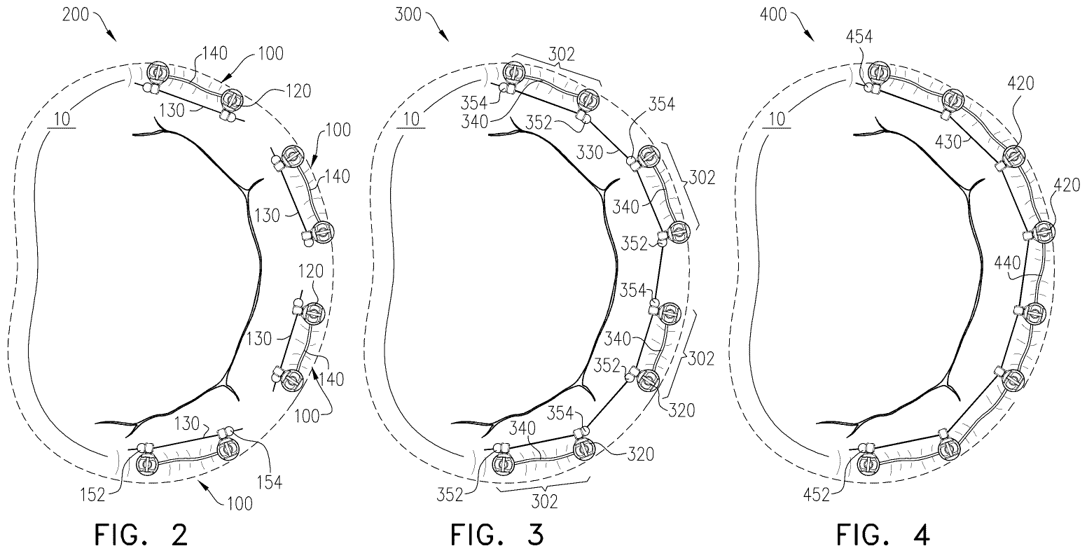

FIG. 3 shows an example implant 300 comprising multiple anchors 320, and a single tether 330 extending between the anchors. In some implementations, implant 300 comprises multiple shape-memory bands 340, each shape-memory band extending between a respective pair 302 of anchors 320, e.g., anchors of each pair adjacent to each other within the implant.

In some implementations, each band 340 extends alongside tether 330. In some implementations, a respective band 340 extends between every anchor and its adjacent anchors, e.g., each pair 302 is connected to another pair via a shape memory band (e.g., bands 340 are present throughout the entire length of the implant).

However, in some implementations, and as shown, each pair 302 is not connected to another pair (e.g., to an adjacent pair) via a shape-memory band. That is, there is a “band-free gap” between bands 340. Nonetheless, as shown, each pair 302 is typically connected to its adjacent pairs via tether 330 (e.g., the tether extends throughout the entire length of the implant).

Similarly to as described for implant 100, at least one anchor 320 of each pair 302 may comprise a terminal 148, via which electrical energy can be applied to the band 340 of the pair.

Implant 300 may comprise multiple pairs of stoppers 352 and 354, that are positioned on tether 330, e.g., on either side of each pair 302 of anchors (e.g., as shown). After each pair 302 of anchors 320 is anchored, that portion of implant 300 is contracted (e.g., as described with reference to FIGS. 1A-E, mutatis mutandis). That is, electrical energy may be applied to the band 340 between the just-deployed pair, and the contraction is locked using a stopper 354.

In some implementations, the portion of the implant at each pair 302 is contracted prior to anchoring the next pair. That is, the contraction of the implant may be performed iteratively, e.g., during implantation of the implant. It is to be noted that, in such cases, tether 330 is typically not cut until contraction of the entire implant 300 has been completed.

In some implementations, multiple (e.g., all) of the pairs 302 of implant 300 are anchored, and then are subsequently contracted in concert.

In some implementations, stoppers are not locked onto both sides of all pairs of anchors, rather a user can select how many anchors, or pairs of anchors, to deploy prior to applying a stopper 354 to lock tension in that segment of the implant. Thus, in some implementations, the tether can be, in effect, divided into segments, with each segment having a degree of tension independent of the other segments.

In some implementations, prior to locking tension in a segment of the implant, electrical energy may be applied to at least one band associated with that segment, in order to augment contraction of that segment of the tether.

In some implementations, implant 300 comprises a single pair of stoppers 352 and 354, one of the stoppers being positioned distally to the distalmost anchor and the other of the stoppers being positioned proximally from the proximal-most anchor.

FIG. 4 shows an example implant comprising multiple anchors 420, a single tether 430 extending between the anchors, and a single shape-memory-band 440, extending alongside the tether, also between the anchors.

In some implementations, only a single anchor 420 of the implant comprises a terminal 148, such that electrical energy is applied to shape-memory band 440 via the terminal in order to contract the entire band. This may, for example, be the last of the anchors to be anchored, or the first of the anchors to be anchored.

In some implementations, a single pair of stoppers 452 and 454 can be used to lock the tension in the implant, e.g., by being positioned on tether 430, on either side of the first deployed anchor and the last deployed anchor.

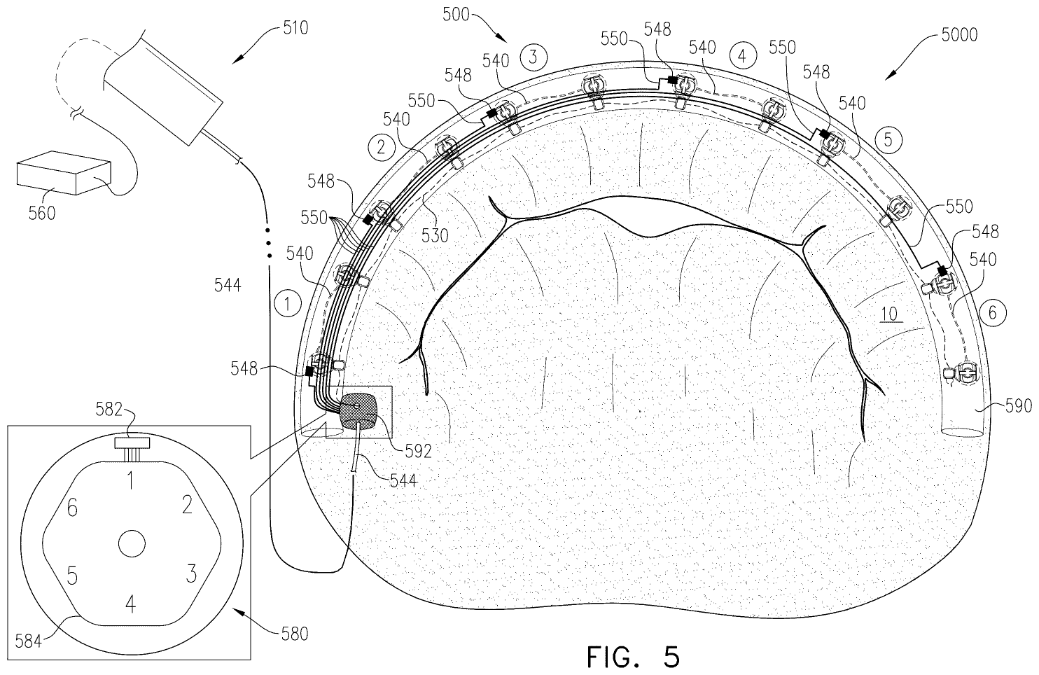

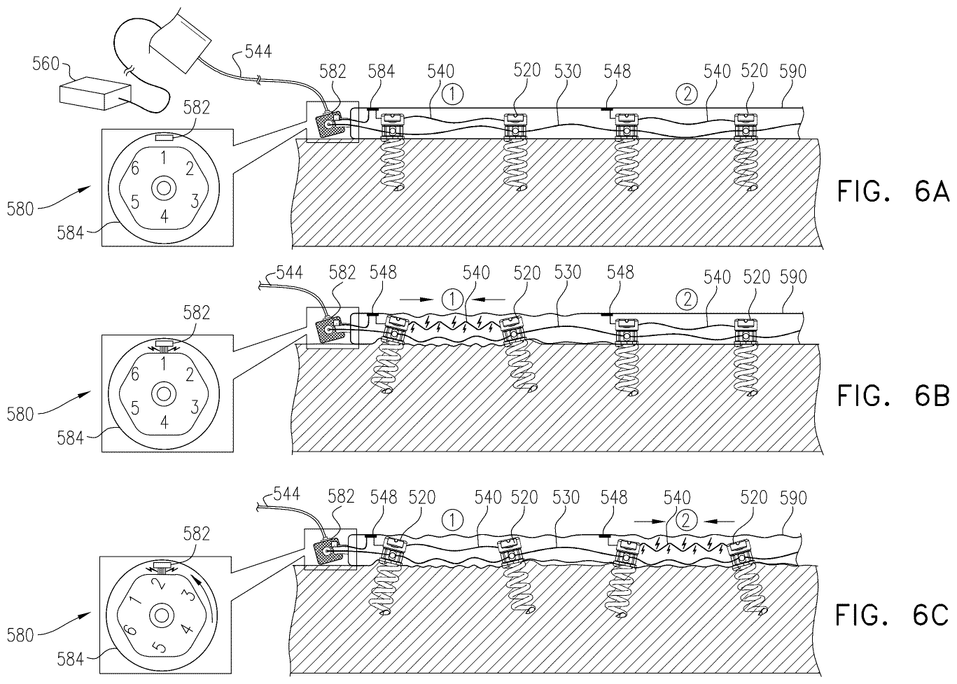

Reference is now made to FIGS. 5 and 6A-C, which illustrate a system 5000 comprising an implant 500 that comprises a hub 580 that is operable by a delivery assembly 510 to select a subset of shape-memory bands of the implant to which to apply energy.

In some implementations, implant 500 is similar to any of implants 200 or 300. For example, implant 500 can comprise multiple anchors 520, and multiple shape-memory bands 540, each shape-memory band extending between a respective subset of anchors, e.g., between anchors that are adjacent to each other within the implant.

In some implementations, implant 500 comprises a single tether 530 that extends between all of the anchors.

In some implementations, implant 500 comprises multiple tethers, each of which extends between the anchors of a corresponding subset. As described hereinabove, in some implementations, each shape-memory band 540 can be contracted (e.g., temporarily) in order to draw the anchors that the shape-memory band connects closer together, in order to facilitate contraction of the implant via tether 530.

Rather than delivery assembly individually engaging each terminal to apply energy to each respective subset of shape-memory bands, delivery assembly 510 can simply engage with hub 580 to electrically connect to each of the shape-memory bands. For example, hub 580 may comprise a hub terminal 582 that a component of the delivery assembly (e.g., a shaft 544, such as a wire or a rod) is adapted to intracardially engage (e.g., while the hub is disposed in the heart), in order to place the delivery assembly in electrical connection with the hub (e.g., as shown in FIG. 5).

In some implementations, hub 580 comprises a switch 584 that is operatable by delivery assembly 510 (e.g., shaft 544 thereof) to select a subset of shape-memory bands to which to electrically connect. In the example shown, switch 584 is a rotary switch, operated by rotation. However, it is to be understood that other switches, electromechanical and/or electronic, may be used. In some implementations, an operator (e.g., a physician) can simply rotate shaft 544 within the switch to rotate the dial, thereby selecting the appropriate subset of shape-memory bands to contract.

In some implementations, system 5000 can comprise a power generator 560, which can be a variant of, or substantively identical to, power generator 160, via which the electrical energy is applied to the shape-memory bands.

In some implementations, each shape-memory band 540, or each subset of shape-memory bands, can comprise a terminal 548 via which delivery assembly 510 is adapted to apply the energy. In some implementations, each terminal 548 can be electrically connected to hub 580 via a corresponding wire 550. In some implementations, each wire 550 can be electrically isolated from the other wires. In some implementations, a shape-memory band 540, or a subset of shape-memory bands, can be contracted by selecting the corresponding position on switch 584 (e.g., using shaft 544) to electrically connect power generator 560 to the corresponding terminal(s) of the shape-memory band(s).

FIGS. 6A-C illustrate an example sequence of application of energy to various shape-memory bands 540 of implant 500. As shown in FIG. 6A, in some implementations, power generator 560 can be electrically connected to the hub by delivery assembly 510 (e.g., shaft 544 thereof) being engaged within hub terminal 582 (e.g., delivery assembly 510 may include a wire that provides electrical conduction).

In some implementations, implant 500 can be delivered to the heart with shaft 544 already engaged with hub 580. In some implementations, shaft 544 can engage hub terminal 582 intracardially, e.g., after the implant has already been delivered to the heart.

FIG. 6B shows an example subset of shape-memory bands (e.g., a single band 540) being contracted by application of energy to that band. As illustrated by the transition between FIG. 6B to 6C, switch 584 is then rotated, and electrical energy is then applied to a different subset of shape-memory bands. This may advantageously allow for the implant to be effectively divided into segments, with each segment having a degree of tension independent of the other segments. For example, in implementations in which implant 500 comprises a tether 530, the tether can be contracted during (and/or after) application of energy to a segment of the implant (e.g., to a subset of shape-memory bands within that segment), which may advantageously allow that segment of the tether to be contracted to a different degree of tension than other segments of the tether.

Although switch 584 is shown having six discrete positions, it is to be understood that any other number of positions are also possible. For example, switch 584 may have 2 positions, 3 positions, or at least 4 positions, and/or fewer than 20 positions, e.g., 4-20 positions. Each position can electrically connect delivery assembly 510 (e.g., power generator 560) to a single shape-memory band, and/or to a subset of shape-memory bands. For example, some positions on switch 584 may connect the delivery assembly to a single shape-memory band, and other positions may connect the delivery assembly to multiple (e.g., combinations of) shape-memory bands. An operator can thus be able to intra-procedurally decide which subsets of shape-memory bands to contract. For example, the operator may wish to contract shape-memory bands that are more closely aligned with the anteroposterior axis of a valve being treated (e.g., bands 1 and 6 in the example shown in FIG. 5)—e.g., in order to draw the anterior and posterior leaflets toward each other.

In some implementations, delivery assembly 510 is adapted to iteratively contract shape-memory bands 540, e.g., by rotating switch 584 and applying small bursts of current to each shape-memory band.

FIG. 5 illustrates an implementation in which implant 500 is an annuloplasty implant comprising a sleeve 590 that is implantable circumferentially along a valve annulus. In some implementations, implant 500 comprises a spool 592 that can be actuated (e.g., rotated) to adjust the tension in tether 530, e.g., to contract sleeve 590 to contract the tissue. In some implementations, and as shown in FIG. 5, spool 592 can be housed within (or co-housed with) hub 580. In some implementations, in addition to placing the delivery assembly in electrical connection with hub 580, shaft 544 can additionally be used to contract tether 530. For example, in the implementation shown, shaft 544 may also be used to actuate (e.g., to rotate) the spool.

In some implementations, sleeve 590 and/or implant 500 is a variant of the sleeves and/or implants as described in US Patent Application Publication 2015/0272734 to Sheps et al., and/or US Patent Application Publication 2018/0049875 to Iflah et al., each of which is incorporated by reference. Although implant 500 is shown as being an annuloplasty implant comprising a sleeve, it is to be understood that an annuloplasty implant not comprising a sleeve could similarly be used, e.g., such as any of those described in US Patent Application Publication 2021/0145584 to Kasher et al., International Patent Application Publication WO 2022/064401 to Halabi et al., and/or International Patent Application Publication WO 2022/172149 to Shafigh et al., each of which is incorporated herein by reference. Furthermore, implant 500 may be an implant other than an annuloplasty implant.

Reference is again made to FIGS. 1A-6C. In some implementations, rather than applying electrical energy to the shape-memory band(s) via a wired connection (e.g., via a conductor, such as conductor 144), wireless energy is applied to the subject (e.g., from outside of the subject, or from within the heart), which in turn causes the shape-memory band(s) to assume its contracted state(s). In some implementations, terminal(s) 148 may not be required.

In some implementations in which multiple shape-memory bands are used, applying wireless energy can cause all of the bands to contract simultaneously. Alternatively, in some implementations, the bands can be contracted sequentially (e.g., one at a time). This can be achieved by positioning a transmitter of wireless energy (e.g., an induction coil) close to each band sequentially, e.g., within the heart, and/or by configuring each band to be responsive to a different frequency of wireless energy.

In some implementations, the wireless energy can be in the form of a wireless RF (Radio Frequency) field.

In some implementations, a receiver coil (not shown) can be included in the head of at least one of the anchors (e.g., at least one anchor of each pair). Such a coil receives the wireless energy and transfers it to the shape-memory band, thereby heating the shape-memory band.

The concepts described above can be applied to other types of implants and/or devices mutatis mutandis. For example, the concepts above can be applied to any implants and/or devices that comprise one or more tethers between two or more components, e.g., similar shape memory and/or electrically responsive materials or bands can be integrated with and/or aligned with the one or more tethers to allow cinching as discussed above.

In some implementations, the concepts herein can be applied to an edge-to-edge repair implant where a tether connects leaflet anchors and the concepts herein can be used to cinch and/or move the leaflet anchors closer together (e.g., with a shape member and/or electrically responsive material or band along the tether), e.g., as described in U.S. Patent Application No. 63/420,440 to Bloodworth et. al., entitled “Heart valve repair devices and methods” and filed on Oct. 28, 2022, which is incorporated by reference herein for all purposes).

In some implementations, the concepts herein can be applied to an implant useable for leaflet repair (e.g., to perform plication, prolapse treatment, or another treatment) where a tether connects leaflet anchors and the concepts herein can be used to cinch and/or move the leaflet anchors closer together (e.g., with a shape member and/or electrically responsive material or band along the tether) to treat a leaflet and/or valve, such as for example, in International Patent Publication WO 2022/250983, which is incorporated by reference herein for all purposes.

### EXAMPLE APPLICATIONS (SOME NON-LIMITING EXAMPLES OF THE CONCEPTS HEREIN ARE RECITED BELOW)

Example 1. A system useable and/or for use at a real or simulated heart, the system comprising: (A) an implant, the implant comprising: (i) a first anchor and a second anchor, (ii) a tether extending between the first anchor and the second anchor, and/or (iii) a shape-memory band that extends alongside the tether, between the first anchor and the second anchor, and/or (B) a delivery assembly, adapted to contract the implant at the heart, by: (i) applying energy to the shape-memory band, such that the shape-memory band draws the first anchor and the second anchor closer together, and/or (ii) securing tension in the implant by locking a stopper to the tether.

Example 2. The system according to example 1, wherein the implant is an annuloplasty implant.

Example 3. The system according to any one of examples 1-2, wherein each of the first anchor and the second anchor comprises a helical tissue-engaging element.

Example 4. The system according to any one of examples 1-3, wherein the delivery assembly comprises an anchor driver, adapted to drive the first anchor into tissue of the heart.

Example 5. The system according to any one of examples 1-4, wherein the shape-memory band is a nitinol band.

Example 6. The system according to any one of examples 1-5, wherein the shape-memory band is fixed to both the first anchor and the second anchor.

Example 7. The system according to any one of examples 1-6, wherein the first anchor is drivable into tissue of the heart while the second anchor is disposed within the delivery assembly, at the heart.

Example 8. The system according to any one of examples 1-7, wherein the implant is a first implant, the system further comprises a second implant, and the delivery assembly is configured to arrange the first implant and the second implant around an annulus of the heart.

Example 9. The system according to any one of examples 1-8, wherein: (A) the first anchor and the second anchor are anchors of a plurality of anchors of the implant, and/or (B) both the tether and the shape-memory band extend between the plurality of anchors.

Example 10. The system according to any one of examples 1-9, wherein the stopper is a bead, slidable over and along the tether.

Example 11. The system according to example 10, wherein the delivery assembly comprises a locking tool configured to advance the stopper over and along the tether.

Example 12. The system according to any one of examples 1-11, wherein the shape-memory band is heat-set to assume a contracted shape upon application of the energy.

Example 13. The system according to example 12, wherein the contracted shape is a zigzag shape.

Example 14. The system according to example 12, wherein the contracted shape is a serpentine shape.

Example 15. The system according to example 12, wherein the contracted shape is a helical shape.

Example 16. The system according to example 12, wherein the shape-memory band is adapted to return toward a relaxed state responsively to cessation of application of the energy to the shape-memory band.

Example 17. The system according to any one of examples 1-16, wherein: (A) the first anchor and the second anchor are a first pair of anchors, the implant comprising multiple pairs of anchors, (B) the shape-memory band is a first shape-memory band, the implant comprising multiple shape-memory bands, each of the shape-memory bands connecting the anchors of a respective pair, and/or (C) the tether extends between all of the anchors of the multiple pairs.

Example 18. The system according to example 17, wherein the implant defines a band-free gap in which no shape-memory band connects the first pair of anchors with a second pair of anchors, the second pair being adjacent to the first pair within the implant.

Example 19. The system according to example 17, wherein: (A) the stopper is a first stopper of a plurality of stoppers, and/or (B) the delivery assembly is adapted to iteratively secure tension in the implant by, for each pair of anchors: (i) applying the energy to the shape-memory band that connects the anchors of the pair, and/or (ii) securing tension in a segment of the tether between the anchors of the pair by locking a respective stopper of the plurality of stoppers to the tether.

Example 20. The system according to example 17, wherein the delivery assembly is configured to apply the energy to each of the shape-memory bands wirelessly.

Example 21. The system according to any one of examples 1-20, wherein the shape-memory band is heat-set to transition toward a contracted shape at a temperature greater than Example 37.5 degrees Celsius.

Example 22. The system according to example 21, wherein the shape-memory band is heat-set to transition toward the contracted shape at a temperature greater than 45 degrees Celsius.

Example 23. The system according to example 22, wherein the shape-memory band is heat-set to transition toward the contracted shape at a temperature greater than 50 degrees Celsius.

Example 24. The system according to example 22, wherein the shape-memory band is heat-set to transition toward the contracted shape at a temperature less than 60 degrees Celsius.

Example 25. The system according to any one of examples 1-24, wherein: (A) for each of the first anchor and the second anchor, the anchor comprises an anchor head, and a collar that circumscribes and is rotatable about the anchor head, and/or (B) the shape-memory band is attached to the collar of the first anchor and the collar of the second anchor.

Example 26. The system according to example 25, wherein each collar defines an eyelet, and wherein the tether extends through the eyelet of the first anchor and the eyelet of the second anchor.

Example 27. The system according to example 26, wherein the tether is slidable through the eyelet of the second anchor.

Example 28. The system according to example 25, wherein the first anchor comprises a terminal on the collar of the first anchor, electrically connectable to the delivery assembly, and configured to conduct the energy from the delivery assembly to the shape-memory band.

Example 29. The system according to any one of examples 1-28, wherein the delivery assembly comprises a catheter that is configured to transluminally deliver the implant to the heart.

Example 30. The system according to example 29, wherein the catheter is configured to transfemorally deliver the implant to the heart.

Example 31. The system according to any one of examples 1-30, wherein the first anchor comprises a terminal, electrically connected to the shape-memory band, and via which the delivery assembly is configured to apply the energy to the shape-memory band.

Example 32. The system according to example 31, wherein: (A) the first anchor comprises a tissue-engaging element, configured to anchor the first anchor to the tissue by being driven into tissue of the heart, and/or (B) the terminal is electrically isolated from the tissue-engaging element.

Example 33. The system according to example 31, wherein: (A) the system further comprises an extracorporeal power generator, and a conductor adapted to electrically connect the terminal to the power generator, and/or (B) the power generator is adapted to apply the energy to the shape-memory band via electrical connection between the conductor and the terminal.

Example 34. The system according to example 33, wherein the conductor is adapted to extend, from the terminal, through the delivery assembly, and out of the subject, where the conductor is connected to the power generator.

Example 35. The system according to any one of examples 1-34, wherein: (A) the delivery assembly further comprises a wireless transmitter, adapted to transmit the energy wirelessly, and/or (B) the implant comprises a receiver, adapted to receive the wireless energy and to transfer at least part of the energy to the shape-memory band.

Example 36. The system according to example 35, wherein the wireless transmitter is adapted to transmit the energy electromagnetically.

Example 37. The system according to any one of examples 1-36, wherein: (A) the first anchor and the second anchor are a first pair of anchors, the implant comprising multiple pairs of anchors, (B) the shape-memory band is a first shape-memory band, the implant comprising multiple shape-memory bands, each of the shape-memory bands connecting the anchors of a respective pair, and/or (C) the implant further comprises a hub with which the delivery assembly is engageable, and that comprises a switch that is operable by the delivery assembly to select a subset of the shape-memory bands to which to apply the energy.

Example 38. The system according to example 37, wherein the subset of shape-memory bands comprises only a single shape-memory band.

Example 39. The system according to example 37, wherein the subset of shape-memory bands comprises multiple shape-memory bands.

Example 40. The system according to example 37, wherein the hub comprises a hub terminal, the delivery assembly adapted to engage the hub terminal in a manner that places the delivery assembly in electrical connection with the hub.

Example 41. The system according to example 37, wherein the delivery assembly is adapted to operate the switch by rotating the switch.

Example 42. The system according to example 37, wherein the switch is a rotary switch.

Example 43. The system according to example 42, wherein the rotary switch has at least four switching positions.

Example 44. The system according to example 37, wherein: (A) for each subset of shape-memory bands, the implant comprises a respective terminal, electrically connected to the respective subset, (B) each terminal is electrically connected to the switch via a corresponding wire, and/or (C) operation of the switch to select the subset electrically connects the delivery assembly to the respective terminal of the subset.

Example 45. The system according to example 44, further comprising an extracorporeal power generator, adapted to apply the energy to the subset of shape-memory bands via electrical connection between the power generator and the respective terminal of the subset.

Example 46. The system according to example 44, wherein each wire is electrically isolated from the other wires.

Example 47. The system according to example 44, wherein the delivery assembly is adapted to iteratively contract each subset by sequentially delivering a burst of energy to each subset.

Example 48. A method useable and/or for use at a real or simulated heart, the method comprising: (A) anchoring an implant to real or simulated tissue of the heart by anchoring a first anchor of the implant to the tissue, and a second anchor of the implant to the tissue, a tether of the implant extending between the first anchor and the second anchor, (B) subsequently, reducing a distance between the first anchor and the second anchor by applying energy to a shape-memory band that extends, alongside the tether, between the first anchor and the second anchor, and/or (C) subsequently, fixing the reduced distance by locking a stopper to the tether.

Example 49. The method according to example 48, further comprising ceasing to apply the energy subsequently to fixing the reduced distance.

Example 50. The method according to any one of examples 48-49, wherein reducing the distance comprises reducing the distance without applying tension to the tether.

Example 51. The method according to any one of examples 48-50, wherein the implant is an annuloplasty implant, and wherein anchoring the first anchor and the second anchor to tissue of the heart comprises anchoring the first anchor and the second anchor to tissue of an annulus of the heart.

Example 52. The method according to any one of examples 48-51, wherein applying the energy to the shape-memory band comprises transmitting the energy to the implant wirelessly.

Example 53. The method according to any one of examples 48-52, wherein applying the energy to the shape-memory band comprises applying the energy to the shape-memory band such that the shape-memory band becomes heated to a temperature greater than Example 37.5 degrees C.

Example 54. The method according to any one of examples 48-53, wherein anchoring the first anchor and the second anchor to tissue of the heart comprises driving a tissue-engaging element of the first anchor and a tissue-engaging element of the second anchor into the tissue.

Example 55. The method according to any one of examples 48-54, further comprising sliding the stopper over and along the tether such that the stopper abuts the second anchor.

Example 56. The method according to any one of examples 48-55, wherein anchoring the first anchor to the tissue comprises anchoring the first anchor to the tissue whilst the second anchor is disposed within a distal part of a delivery assembly that is disposed within the heart.

Example 57. The method according to any one of examples 48-56, wherein the method further comprises, subsequently to locking the stopper to the tether, trimming an excess of the tether.

Example 58. The method according to any one of examples 48-57, wherein the implant is a first implant, and wherein the method comprises implanting multiple implants in an arrangement along the tissue.

Example 59. The method according to any one of examples 48-58, wherein: (A) the first anchor and the second anchor are anchors of a plurality of anchors of the implant, and/or (B) anchoring the implant to the tissue comprises anchoring the implant along the tissue while both the tether and the shape-memory band extend between the plurality of anchors.

Example 60. The method according to any one of examples 48-59, wherein: (A) the implant includes multiple pairs of anchors, the first anchor and the second anchor being one of the pairs of anchors, (B) the shape-memory band is a first shape-memory band, the implant comprising multiple shape-memory bands, each of the shape-memory bands connecting the anchors of a respective pair, (C) the implant further includes a hub that comprises a switch, (D) the method further comprises, prior to applying the energy to the first shape-memory band, selecting a subset of the multiple shape-memory bands by operating the switch, the subset including the first shape-memory band, and/or (E) reducing the distance between the first anchor and the second anchor by applying energy to the first shape-memory band comprises reducing the distance between the first anchor and the second anchor by applying the energy to the selected subset of shape-memory bands.

Example 61. The method according to example 60, wherein the switch is a rotary switch, and wherein operating the switch comprises rotating the rotary switch.

Example 62. The method according to any one of examples 48-61, further comprising applying tension to the tether prior to locking the stopper to the tether.

Example 63. The method according to example 62, wherein applying the tension to the tether comprises applying the tension to the tether subsequently to commencing applying the energy to the shape-memory band.

Example 64. The method according to any one of examples 48-63, wherein: (A) the first anchor and the second anchor are a first pair of anchors, the implant comprising multiple pairs of anchors, (B) the shape-memory band is a first shape-memory band, the implant comprising multiple shape-memory bands, each of the shape-memory bands connecting the anchors of a respective pair, and/or (C) the method comprises implanting the implant along the tissue while the tether extends between all of the anchors of the multiple pairs.

Example 65. The method according to example 64, wherein anchoring the implant to the tissue comprises anchoring the implant to the tissue in a manner that defines a band-free gap in which no shape-memory band connects the first pair of anchors with a second pair of anchors of the implant, the second pair being adjacent to the first pair within the implant.

Example 66. The method according to example 64, wherein: (A) the stopper is a first stopper of a plurality of stoppers, and/or (B) the method comprises iteratively securing tension in the implant by, for each pair of anchors: (i) applying the energy to the shape-memory band that connects the anchors of the pair, and/or (ii) securing tension in a segment of the tether between the anchors of the pair by locking a respective stopper of the plurality of stoppers to the tether.

Example 67. The method according to any one of examples 48-66, wherein the method comprises transluminally delivering the implant to the heart.

Example 68. The method according to example 67, wherein transluminally delivering the implant to the heart comprises transfemorally delivering the implant to the heart.

Example 69. The method according to any one of examples 48-68, wherein applying the energy to the shape-memory band comprises applying the energy to the shape-memory band such that shape-memory band transitions toward a contracted shape.

Example 70. The method according to example 69, wherein the method further comprises, subsequently to locking the stopper to the tether, ceasing to apply the energy to the shape-memory band, such that the shape-memory band returns toward a relaxed state.

Example 71. The method according to any one of examples 48-70, wherein the method is performed on a simulation.

Example 72. The method according to any one of examples 48-70, wherein the method further comprises sterilizing the implant prior to anchoring the implant to tissue of the heart.

Example 73. A method useable and/or for use at a real or simulated heart, the method comprising: (A) implanting an implant at the heart by securing a plurality of anchors of the implant to real or simulated tissue of the heart, the anchors connected together via (i) a shape-memory band, and (ii) a tether, (B) transitioning the implant towards a contracted state by applying electrical energy to the shape-memory band, such that the anchors move closer together, and/or (C) locking the implant in the contracted state by applying a stopper to the tether.

Example 74. The method according to example 73, wherein the method is performed on a simulation.

Example 75. The method according to example 73, wherein the method further comprises sterilizing the implant prior to anchoring the implant to tissue of the heart.

Example 76. A method useable and/or for use at a real or simulated heart, the method comprising: (A) anchoring, to tissue of the heart, a first anchor and a second anchor, a portion of a tether extending between the first anchor and the second anchor, the portion having a length, (B) subsequently, reducing the length of the portion of the tether, facilitated by applying electrical energy to a shape-memory band that extends, alongside the tether, between the first anchor and the second anchor, and/or (C) subsequently, fixing the reduced length by locking a stopper to the tether.

Example 77. The method according to example 76, wherein the method is performed on a simulation.

Example 78. The method according to example 76, wherein the method further comprises sterilizing the first anchor and the second anchor prior to anchoring.

Example 79. An apparatus comprising an implant comprising: (A) multiple shape-memory bands, configured such that application of energy to a shape-memory band contracts a respective portion of the implant, and/or (B) a hub comprising: (i) a hub terminal, via which the energy is applied, and/or (ii) a switch that is operable to select a shape-memory band to which to direct the energy.

Example 80. The apparatus according to example 79, wherein the apparatus further comprises a delivery assembly configured to deliver the implant into a subject, and comprising a shaft that is adapted to: (A) engage the hub terminal in a manner that places the delivery assembly in electrical connection with the hub, and/or (B) operate the switch to select the shape-memory band.

Example 81. The apparatus according to example 80, wherein the shaft is flexible, and is adapted to be transluminally advanced to a tissue of a subject.

Example 82. The apparatus according to example 80, wherein: (A) the multiple shape-memory bands are grouped into multiple subsets of shape-memory bands, (B) for each subset of shape-memory bands, the implant comprises a respective terminal, electrically connected to the respective subset, (C) each terminal is electrically connected to the switch via a corresponding wire, and/or (D) operation of the switch to select the subset electrically connects the shaft to the respective terminal of the subset.

Example 83. The apparatus according to example 82, further comprising an extracorporeal power generator, electrically connected to the shaft, and adapted to apply the energy to the subset of shape-memory bands via the electrical connection between the shaft and the respective terminal of the subset.

Example 84. The apparatus according to example 82, wherein each wire is electrically isolated from the other wires.

Example 85. The apparatus according to any one of examples 79-84, wherein the switch is a rotary switch.

Example 86. The apparatus according to example 85, wherein the rotary switch has at least four switching positions.

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

The techniques, methods, operations, steps, etc. described or suggested herein or in the references incorporated herein, and any methods of using the systems, assemblies, apparatuses, devices, etc. herein, can be performed on a living subject (e.g., human, other animal, etc.) or on a simulation (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

The present invention is not limited to the examples that have been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A system for use at a heart of a subject, the system comprising:
an implant, the implant comprising:
a first anchor and a second anchor;
a tether extending between the first anchor and the second anchor; and
a shape-memory band that extends alongside the tether, between the first anchor and the second anchor; and
a delivery assembly, adapted to contract the implant at the heart, by:
applying energy to the shape-memory band, such that the shape-memory band draws the first anchor and the second anchor closer together, and
securing tension in the implant by locking a stopper to the tether.

2. The system according to claim 1, wherein the implant is an annuloplasty implant.

3. The system according to claim 1, wherein each of the first anchor and the second anchor comprises a helical tissue-engaging element.

4. The system according to claim 1, wherein the first anchor is drivable into tissue of the heart while the second anchor is disposed within the delivery assembly, at the heart.

5. The system according to claim 1, wherein:
the first anchor and the second anchor are a first pair of anchors, the implant comprising multiple pairs of anchors,
the shape-memory band is a first shape-memory band, the implant comprising multiple shape-memory bands, each of the shape-memory bands connecting the anchors of a respective pair, and
the tether extends between all of the anchors of the multiple pairs.

6. The system according to claim 5, wherein the implant defines a band-free gap in which no shape-memory band connects the first pair of anchors with a second pair of anchors, the second pair being adjacent to the first pair within the implant.

7. The system according to claim 5, wherein:
the stopper is a first stopper of a plurality of stoppers, and
the delivery assembly is adapted to iteratively secure tension in the implant by, for each pair of anchors:
applying the energy to the shape-memory band that connects the anchors of the pair, and
securing tension in a segment of the tether between the anchors of the pair by locking a respective stopper of the plurality of stoppers to the tether.

8. The system according to claim 5, wherein the delivery assembly is configured to apply the energy to each of the shape-memory bands wirelessly.

9. The system according to claim 1, wherein:
for each of the first anchor and the second anchor, the anchor comprises an anchor head, and a collar that circumscribes and is rotatable about the anchor head, and
the shape-memory band is attached to the collar of the first anchor and the collar of the second anchor.

10. The system according to claim 9, wherein each collar defines an eyelet, and wherein the tether extends through the eyelet of the first anchor and the eyelet of the second anchor.

11. The system according to claim 9, wherein the first anchor comprises a terminal on the collar of the first anchor, electrically connectable to the delivery assembly, and configured to conduct the energy from the delivery assembly to the shape-memory band.

12. The system according to claim 1, wherein the delivery assembly comprises a catheter that is configured to transluminally deliver the implant to the heart.

13. The system according to claim 1, wherein the first anchor comprises a terminal, electrically connected to the shape-memory band, and via which the delivery assembly is configured to apply the energy to the shape-memory band.

14. The system according to claim 13, wherein:
the first anchor comprises a tissue-engaging element, configured to anchor the first anchor to the tissue by being driven into tissue of the heart, and
the terminal is electrically isolated from the tissue-engaging element.

15. The system according to claim 13, wherein:
the system further comprises an extracorporeal power generator, and a conductor adapted to electrically connect the terminal to the power generator, and
the power generator is adapted to apply the energy to the shape-memory band via electrical connection between the conductor and the terminal.

16. The system according to claim 15, wherein the conductor is adapted to extend, from the terminal, through the delivery assembly, and out of the subject, where the conductor is connected to the power generator.

17. The system according to claim 1, wherein:
the delivery assembly further comprises a wireless transmitter, adapted to transmit the energy wirelessly, and
the implant comprises a receiver, adapted to receive the wireless energy and to transfer at least part of the energy to the shape-memory band.

18. The system according to claim 17, wherein the wireless transmitter is adapted to transmit the energy electromagnetically.

19. The system according to claim 1, wherein:
the first anchor and the second anchor are a first pair of anchors, the implant comprising multiple pairs of anchors,
the shape-memory band is a first shape-memory band, the implant comprising multiple shape-memory bands, each of the shape-memory bands connecting the anchors of a respective pair, and
the implant further comprises a hub with which the delivery assembly is engageable, and that comprises a switch that is operable by the delivery assembly to select a subset of the shape-memory bands to which to apply the energy.

20. The system according to claim 1, wherein the delivery assembly comprises a locking tool configured to transluminally advance the stopper over and along the tether toward the heart.
